(19) United States
(12) Patent Application Publication
(10) Pub. No.: US 2012/0272980 A1
(43) Pub. Date: Nov. 1, 2012

(54) METHOD OF APPLYING MAKEUP TO KERATINOUS FIBERS

(75) Inventors: Henri Samain, Bievres (FR); Christophe Dumousseaux, Antony (FR); Carole Le Merrer, Montrouge (FR)

(73) Assignee: L'OREAL, Paris (FR)

(21) Appl. No.: 13/378,569

(22) PCT Filed: Jun. 18, 2010
(86) PCT No.: PCT/IB2010/052771

§ 371 Date: Jul. 18, 2012

Related U.S. Application Data
(60) Provisional application No. 61/213,964, filed on Aug. 3, 2009.

(30) Foreign Application Priority Data
Jun. 19, 2009 (FR) 0954152

(51) Int. Cl. A61K 8/02; A45D 44/00

(52) U.S. Cl. 132/200; 132/317

(57) ABSTRACT

The present invention relates to a method of applying makeup to human keratinous fibers, in particular the eyelashes, said method comprising the steps consisting in: a) applying on said fibers, an adhesive first composition that forms a discontinuous deposit on the fibers; and b) applying on the fibers coated in this way, a second composition in the loose powder state.

The present invention relates to applying makeup to macroscopic human keratinous fibers, and more particularly, but not exclusively, to applying makeup to the eyelashes, the eyebrows, or the hair.

Most cosmetic compositions for the eyelashes have the function of coloring them and of thickening them. The eyelashes thus become blacker and thicker and are therefore more visible to the consumer or to an outside observer. However, this visibility remains insufficient with conventional mascaras and lacks shine.

The introduction of nacres into most makeup compositions, in particular into mascaras, does not necessarily improve shine and visibility. Since nacres are mixed with other components of the formulation, such as waxes or polymers, their shine may be diminished or even lost.

It has been suggested to use a two-component makeup system for improving the staying power or the optical effects of cosmetic compositions.

U.S. Pat. No. 6,367,484 describes using a first composition containing a film-forming polymer, and depositing solid particles belonging to a second composition before the first composition has dried completely. The solid particles are preferably spheres and the desired effect is preferably a relief effect. Examples of nail varnish are described. The effect obtained is difficult to remove and is very sensitive to drying time.

Application WO 02/05762 describes using a first composition containing an adhesive material, and then applying a second cosmetic composition. The first composition imparts an improved staying power effect. The examples mention using polyisobutylenes in the first composition, and the second composition is in powder form.

Examples of compositions including adhesive particles are described in US 2006/0024255, US 2002/0041858, and US 2007/0224140.

There exists a need to create, in easy manner, attractive visual effects on keratinous fibers, in particular the eyelashes, that are both long lasting and easy to remove.

The invention seeks to satisfy this need, and in first exemplary embodiments, it achieves this by means of a method of applying makeup to human keratinous fibers, in particular the eyelashes, said method comprising the steps consisting in:

1) applying on said fibers, an adhesive first composition that forms a discontinuous deposit on the fibers;

2) applying on the fibers coated in this way, a second composition in the loose powder state.

The invention may make it easy to obtain a relatively large deposit of particles of the second composition, leading to visual effects that may be very visible, e.g. shiny, filling, and/or lengthening effects.

In addition, the makeup effect is long lasting, and removing it remains easy.

The particles of the second composition may be particles having one or more pigments. Particles in the form of flakes are not essential, but are merely preferred. The particles may be fibers, microbeads, or other micro-objects.

### Adhesive Composition

The adhesive first composition includes an adhesive that is adapted to hold the particles of the second composition on the keratin fibers, in particular the eyelashes, coated in the adhesive composition.

In accordance with the invention, the adhesive composition is selected so that it creates a discontinuous adhesive film on the fibers being treated. Thus, the adhesive composition need not include adhesive that forms a continuous film on the fibers being treated.

The Applicant has observed that the presence of a discontinuous film makes it easier to apply and to remove makeup, and, from an appearance point of view, may lead to better results than when using an adhesive composition that forms a continuous film on the surface of the fibers. The discontinuous film decreases the risk of the eyelashes sticking together, and may make it easier to separate the eyelashes while makeup is being applied.

The adhesive composition may include adhesive particles that make it possible to obtain a deposit that retains residual adhesive power after drying and that does not form a continuous film. The adhesive particles may be of various forms and may be of average size lying in the range 1 micrometer (μm) to 100 μm, or even more.

The average size of the adhesive particles may lie in the range 10 μm to 250 μm.

The expression “average size” must be understood as the granulometric statistic size at the half of the population (i.e. D(0.5))

The behavior of these adhesive particles is different from the behavior of adhesive polymers of the polyisobutylene (PIB) or latex type which, on drying, form liquid or dry films. In spite of their adhesive power, the adhesive particles do not coalesce. It is possible to observe particles that are stuck together, but each particle maintains all or some of its individuality. The use of such adhesive particles makes it easier to apply and to remove makeup.

The obtaining of a discontinuous deposit is, when the adhesive composition includes adhesive particles, linked to the fact that the adhesive particles are deposited in a discontinuous manner on the fibers.

Within an adhesive composition of the invention, the adhesive particles may be present at a concentration lying in the range 0.1% to 70%, preferably in the range 0.5% to 10%, more preferably in the range 1% to 5%.

The adhesive composition may be in the form of a gel or of an emulsion, preferably being relatively fluid, with a volatile phase enabling drying.

However, the adhesive composition may form, once deposited on the keratin fibers, a discontinuous deposit of adhesive particles that are not fluid.

The expression “adhesive particles that are not fluid” means that the adhesive particles have a viscosity such that they do not flow at room temperature (20° C.) under the only effect of gravity when deposited on the keratin fibers.

The solvent content may be greater than or equal to 50% by weight, preferably greater than 70% by weight.

The adhesive particles may be mixed with other components such as gelling agents or pigments.

The adhesive particles may be made out of a single material or out of two or more materials. In particular, the adhesive particles may present a core made out of a non-adhesive material, e.g. an inorganic or an organic material, and a shell made out of an adhesive coating.

The adhesive material(s) of the adhesive composition, in particular the above-mentioned adhesive particles, are advantageously selected from adhesives that are sensitive to pressure that retain adhesive power after drying.

In particular, the adhesive composition may comprise pressure-sensitive adhesives selected from: acrylic polymers, in particular acrylate and methacrylate copolymers; rubber-based or styrene-copolymer-based pressure-sensitive adhesives, e.g. such as styrene-isoprene-styrene (SIS) and styrene-butadiene-styrene (SBS) copolymers.

The adhesive composition may also comprise pressure-sensitive adhesives selected from: urethane polymers; polyurethanes; cross-linked silicones; ethylene-vinyl acetate polymers; styrene-based block copolymers; or natural gums; chloroprene; butadiene; isoprene; neoprene polymers; or the like.

By way of non-limiting examples of rubber-polymer-based pressure-sensitive adhesives, mention may be made in particular of natural rubber (poly(cis-1,4-isoprene)); methyl-methacrylate-isoprene graft copolymers; styrene butadiene copolymers; butyl rubber; acrylonitrile-butadiene rubber; styrene-isoprene block copolymers; polybutadiene, ethylene-butylene block copolymers; and polychloroprene.

Amongst pressure-sensitive adhesives including polar acrylic polymers, mention may be made of acrylic acid-based block or statistical copolymers; alkyl acrylates and alkyl methacrylates; and copolymers of such acrylics with ethylene and vinyl acetate.

An example of pressure-sensitive adhesive that is particularly suitable is poly(2-ethylhexylacrylate), e.g. sold under the trade name GEL-TAC 100G (Advanced Polymer International), as a 40% aqueous dispersion of solid material comprising 15 μm adhesive acrylic microspheres.

The obtaining of an adhesive discontinuous deposit on the keratin fibers is the consequence of the size of the adhesive particles used. Adhesive particles of too small size may coalesce and form a continuous film when deposited on the keratin fibers. As such, it may be advantageous to use adhesive particles of average size greater than 1 μm, for example lying in the range fpm to 250 μm.

The adhesive composition may be deposited on the eyelashes using any suitable applicator, e.g. of mascara brush or comb type. The adhesive composition may be contained in a container, and the applicator may be arranged to fasten on the container and to close it in leaktight manner. By way of example, the container includes a threaded neck and the applicator includes a handle that is arranged to screw-fasten on the neck and that also constitutes a closure member for closing the container. The applicator may include a stem that carries the applicator element, e.g. a brush having a twisted or injection-molded core. The container may include a wiper member for wiping the stem and the applicator member.

By way of example, the quantity of adhesive composition deposited on the eyelashes lies in the range 5 milligrams (mg) to 50 mg per set of eyelashes.

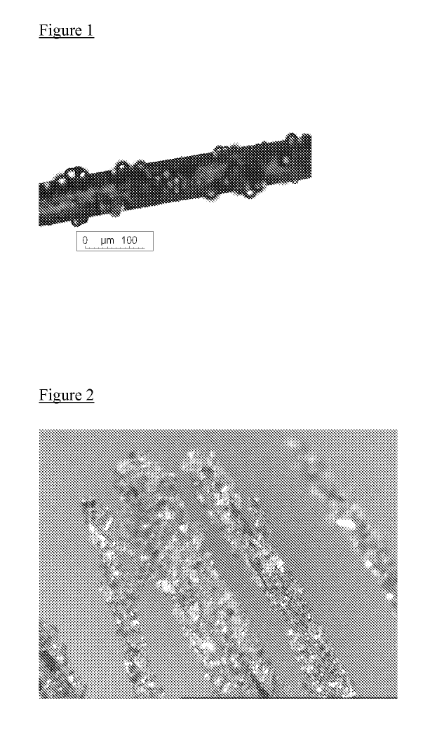

FIG. 1 shows an eyelash coated with an adhesive composition made in accordance with a preferred implementation of the invention. It can be seen that the composition forms a discontinuous deposit of adhesive particles.

### Powder Composition

The second composition may include particles that are flakes, but that may also be in some other form.

The particles may optionally be reflective, depending on the effect desired.

The term “flake” should be understood to mean that the thickness e of the particle is less than its greatest dimension d. By way of example, d/e≧10, or even d/e≧50.

The particles may have at least one plane face, or may have a radius of curvature that is greater than or equal to 60 μm. This may make it easier to stack the particles and to increase their specular reflective power.

A greatest dimension of the particles, whatever their shape, may lie in the range 1 μm to 10 μm, preferably in the range 5 μm to 500 μm, more preferably still in the range 10 μm to 200 μm. The size of the particles is preferably greater than or equal to 10 μm, better greater than or equal to 20 μm, better still greater than or equal to 40 μm.

The form factor of said particles is advantageously greater than or equal to 5, better greater than or equal to 10, better still greater than or equal to 50.

The second composition need not have a liquid binder. Preferably, the quantity of liquid binder is less than 20% by weight, better less than 15%, or even less than 5% by weight, so as not to hinder unduly the fluidity of the second composition. Good fluidity of the powder composition may make it easier to deposit on the eyelashes, and may enable a coating to be formed that is attractive.

The second composition may be reflective.

### Measuring the Reflective Power of the Powder Composition

The composition of reflective power that is to be measured is applied in uniform manner, at a rate of 0.2 milligrams per square centimeter (mg/cm²), on a surface made of black Bioskin®, sold by Beaulax.

The reflective power is measured with the GP-5 goniophotometer sold by Murakami.

The angle of incidence is fixed at −45°, and the reflectance is measured over the range −90° to 90°. The reflectance maximum, corresponding to specular reflectance, is generally measured at 45°, and it is marked R₄₅. The reflectance minimum, corresponding to diffuse reflectance, is generally measured at −30°, and it is marked R₋₃₀.

The reflective power of the composition is defined by R₄₅/R₋₃₀.

The reflective power of the second composition of the invention, whatever the shape of the reflective particles, may preferably be greater than 5, more preferably greater than 7, and better greater than 10.

Values obtained on applying compositions containing 100% of the following particles:

| Particle type | Trade reference | R₄₅/R₋₃₀ |
|---|---|---|
| Spherical | Covabead L85 by LCW | 0.9 |
| polymethyl | | |
| methacrylate | | |
| (PMMA) | | |
| Spherical silica | SB150 by Miyoshi | 1.1 |
| Oblong-shaped | Orgasol 2002 by Arkema | 1.4 |
| nylon ® | | |
| Lamellar talc | Talc P3 by Luzenac | 2.4 |
| Bismuth | BIRON LF 2000 by Merck | 7.4 |
| oxychloride | | |
| Mica nacre - | Prestige Gold by Eckart | 8.1 |
| TiO₂ | | |
| Silica flake | Silkyflake FTD010 by NSG | 8.4 |
| Mica nacre - | Flamenco Green 820C by BASF | 10.1 |
| TiO₂ | | |
| Synthetic mica | PDM-20L by Topy | 14.2 |
| Silver-coated | Metashine ME2040PS by NSG | 56.8 |
| borosilicate | | |

The particles, e.g. reflective particles, in particular particles in the form of flakes, are present at a content that is greater than or equal to 30% by weight, preferably greater than or equal to 50% by weight, better greater than or equal to 70% by weight, and better still greater than or equal to 80% by weight, relative to the total weight of the powder composition.

The powder composition of the invention preferably includes nacres as reflective-flake particles.

The term “nacre” should be understood to mean particles of any shape that may optionally be iridescent, as produced in the shells of certain mollusks, or that are synthesized, and that exhibit a “pearlescent” coloring effect by optical interference. The color effect obtained corresponds to one of the colors of the rainbow, to gold, or to silver.

Two large families may be distinguished within reflective-flake particles:
flake particles having at least two parallel faces that consist of a single material that is optically uniform; and
flake particles that have a layered structure with at least two layers of material that are different optically (pigments having a substrate and coating structure, or pigments that are multilayered without a substrate).

As examples of flake particles having at least two parallel faces that consist of a single material that is optically uniform, mention may be made of: metal-effect pigments, such as metal flakes, e.g. flakes of aluminum or of metal-alloy, e.g. copper-zinc alloy; silica, synthetic mica, or glass particles; or transparent-effect pigments such as crystalline bismuth oxychloride or polycrystalline titanium dioxide.

As examples of metal pigments, mention may be made of aluminum, bronze, or copper powders that are coated with SiO₂ and sold under the trade name VISIONAIRE by ECKART.

As examples of glass flakes, mention may be made of compositions sold under the name SILKYFLAKE by Nippon Sheet Glass.

As an example of bismuth oxychloride-based pigment, mention may be made of BIRON pigments sold by Merck, and PEARL compositions sold by FARMAQUIMIA.

As examples of flake particles having a layered structure, mention may be made of flake particles formed of natural or synthetic mica coated with at least one layer of metal oxide selected, for example, from: oxides of titanium, in particular TiO₂, of iron, in particular Fe₂O₃, of tin, and of chromium; barium sulfate; and the following materials: MgF₂; CrF₃; ZnS; ZnSe; SiO₂; Al₂O₃; MgO; Y₂O₃; SeO₃; SiO; HfO₂; ZrO₂; CeO₂; Nb₂O₅; Ta₂O₅; MoS₂; and their mixtures or alloys.

By way of examples of such particles, mention may be made of particles comprising a substrate of synthetic mica coated with titanium dioxide; and of glass particles coated with brown iron oxide, titanium oxide, tin oxide, or one of their mixtures such as those sold under the trade name REFLECKS® by ENGELHARD.

Mention may also be made of: titanium mica coated with iron oxide; mica coated with bismuth oxychloride; titanium mica coated with chromium oxide; titanium mica coated with an organic colorant; and nacre pigments based on bismuth oxychloride. The term “titanium mica” means mica coated with TiO₂.

They may also be particles of mica on the surface of which at least two successive layers of metal oxides and/or organic coloring materials have been superposed. The nacres may present a yellow, pink, red, bronze, orangey, brown, gold, and/or coppery color or glint.

Illustrative examples of nacres suitable for being introduced into the second composition and that may be mentioned are gold color nacres, in particular those sold by ENGELHARD under the trade names Brillant gold 212G (Timica), Gold 222C (Cloisonne), Sparkle gold (Timica), Gold 4504 (Chromalite), and Monarch gold 233× (Cloisonne); bronze nacres, in particular those sold by MERCK under the trade names Bronze fine (17384) (Colorona) and Bronze (17353) (Colorona), and by ENGELHARD under the trade name Super bronze (Cloisonne); orange nacres especially those sold by ENGELHARD under the trade names Orange 363C (Cloisonne) and Orange MCR 101 (Cosmica), and by MERCK under the trade names Passion orange (Colorona) and Matte orange (17449) (Microna); brown-tinted nacres sold by ENGELHARD under the trade names Nu-antique copper 340XB (Cloisonne) and Brown CL4509 (Chromalite); nacres with a copper glint sold by ENGELHARD under the trade name Copper 340A (Timica); nacres with a red glint, especially those sold by MERCK under the trade name Sienna fine (17386) (Colorona); nacres with a yellow glint, especially those sold by ENGELHARD under the trade name Yellow (4502) (Chromalite); red-tinted nacres with gold glints, especially those sold by ENGELHARD under the trade name Sunstone G012 (Gemtone); pink nacres, especially those sold by ENGELHARD under the trade name Tan opale G005 (Gemtone); black nacres with a glint, especially those sold by ENGELHARD under the trade name Nu antique bronze 240 AB (Timica); blue nacres, especially those sold by MERCK under the trade name Matte blue (17433) (Microna); white nacres with silvery glints, especially those sold by MERCK under the trade name Xirona Silver; and orange, pink, green, and gold highlight nacres sold by MERCK under the trade names Indian summer (Xirona) and mixtures thereof. Mention may also be made of interference particles on a glass base such as Ronastar by MERCK, or on a synthetic-mica base such as Sunshine by SUN CHEMICAL or PROMINENCE by NIKON KOKEN.

As reflective particles, mention may also be made of polyethylene terephthalate (PET) based flakes such as those sold under the reference Disco by GLITTEREX or under the reference MICROGLITTER by VENTURE CHEMICAL, and PET/Nylon® based interference fibers proposed under the reference Morphotone by TEIJIN.

For a multilayer interference structure in particular, the reflective particles may, for example, present at least one layer of uniform thickness, in particular of a reflective material.

When the reflective particles have a multilayer structure they may, for example, comprise a natural or synthetic substrate, in particular a synthetic substrate which is at least partially coated with at least one layer of a reflective material, in particular at least one metal or metallic material. The substrate may be a single material or multiple materials, and it may be organic and/or inorganic. More particularly, it may be selected from glasses, ceramics, graphite, metal oxides, aluminas, silicas, silicates, in particular aluminosilicates and borosilicates, synthetic mica, and mixtures thereof, this list not being limiting.

Reflective particles have been described in particular in Japanese patent documents JP-A-09188830, JP-A-10158450, JP-A-10158541, JP-A-07258460, and JP-A-05017710.

Further examples of reflective particles comprising a mineral substrate coated with a metal layer that may be mentioned are particles comprising a substrate of borosilicate coated with silver, sold by Nippon Sheet Glass under the trade name METASHINE.

The second composition may also include optionally-reflective spherical particles, and oblong particles.

The second composition may include any volatile solvent, e.g. ethanol, acetone, dimethyl ether, alkanes, water, or volatile silicones.

In order to apply the second composition, it is possible to use any applicator that is capable of taking a powder composition, the applicator including a foam or flocked endpiece, for example. The applicator may include a flat endpiece that is circularly-cylindrical or cone shaped.

The fineness of the foam or the thickness of the flocking may be adjusted so as to enable the user to take the optimum quantity of composition.

Where appropriate, the applicator includes a magnetized endpiece, and that may make it easier to take particles having magnetic susceptibility.

In order to apply the powder composition, it is also possible to use a substrate, e.g. in the form of a sheet or of a patch, on which the second composition is already deposited in the form of a powder layer. The substrate may be made out of paper, out of non-woven fabric, or out of plastics material.

It is also possible to use an applicator with suction, e.g. including an endpiece that is coated with a surface that is porous to air and of fineness that prevents particles from passing therethrough. The endpiece is connected to a source of suction, e.g. a pump or a bulb. The user sucks up a small quantity of composition in order to take said composition, and when entering into contact with the eyelashes, decreases or even eliminates the suction so as to enable the composition to be deposited.

Other systems including a vibrator applicator member may be suitable for applying the powder composition.

An applicator having the shape of a set of eyelashes may be used to apply a large quantity of powder in a single movement.

Application may be performed without rubbing the applicator on the fibers.

It is possible to finalize application using a third movement, e.g. for fixing or for removing any excess powder composition.

When the adhesive presents a permanent characteristic, the user may reapply the second composition as desired, without necessarily having to reapply the first composition.

In an implementation of the invention, only the underneath or only the top of the set of eyelashes is treated. This creates a striking optical effect when the person blinks.

In another implementation of the invention, only a limited number of eyelashes within the set are treated, e.g. only one in two.

The user may treat only a portion of the eyelashes, e.g. only the tips or an intermediate portion. The application of the second composition makes it possible to magnify such portions or to make them less visible. For example, it is possible to treat only the roots so as to make them less visible and so as to magnify the tips.

The first composition may be applied widely, and the second composition may be applied in a pattern. A third composition of transparent powder may then be applied on the first two compositions.

Alternatively, the first composition may be applied widely, and another composition of transparent powder may be applied in a pattern. The second composition of the invention is then applied, which composition sticks only onto the zones that have not received the transparent powder composition. On removing the surplus, the pattern appears.

Alternatively, the first composition may be applied in a pattern, then the second composition may be applied in the same zone. The pattern then appears as the particles that are not held by the adhesive are eliminated.

The particles of the second composition may themselves be adhesive.

The first composition may generate particles, e.g. on drying. By way of example, the first composition contains materials that are suitable for crystallizing as waxes, for example.

If necessary, it is possible to add, in the first composition, agents that provide a spreading effect, e.g. a compound of low surface tension and/or an agent that decreases surface tension, so that on a single contact, the first composition may, when deposited, spread over the entire surface of the eyelashes.

It is possible to press the deposit of second composition so as to flatten it.

### Kits

The adhesive composition, the powder composition, and the applicators may be made available to the user within a single package, in the form of a kit.

The kit may include a single adhesive composition and at least two different powder compositions, e.g. including different kinds of particle, e.g. reflective flakes. The compositions may be packaged separately within a single package.

The container containing the adhesive composition and the container containing the powder composition may be connected together, e.g. via their sides or via their bottoms.

The kit may include means that make it possible to remove any surplus of second composition, e.g. by suction, by friction, or by magnetic attraction.

### EXAMPLES

### All of the Proportions Indicated are by Weight

### Example 1

Composition A: wax-in-water mascara formula

| | Composition A |
|---|---|
| Paraffin wax | 12.8 |
| Carnauba wax | 5.0 |
| Beeswax | 4.1 |
| Cetylic alcohol | 2.0 |
| Steareth 20 | 4.4 |
| Steareth 2 | 2.1 |
| Iron oxide | 7.1 |
| Glycerine | 2.0 |
| Butylene glycol | 3.0 |
| Potassium cetyl phosphate | 1.7 |
| Hydroxyethylcellulose | 0.9 |
| Gum arabic | 3.4 |
| Water | Qs 100 |

Composition B: Composition A in Example 1 of application WO 02/05762
Adhesive compositions C and D:

| | Composition C | Composition D |
|---|---|---|
| Roderm 560* | 20 | |
| Gel-Tac 600D** | | 20 |
| Hydroxyethylcellulose | 3 | 3 |
| (HEC) | 77 | 77 |
| Water | | |

*Adhesive acrylic latex sold by Rohm and Haas (55% dry extract, Tg = −30° C.)
**Adhesive particles in aqueous solution sold by Advanced Polymer International (44% dry extract).

Makeup was applied in two stages, i.e. the base composition (A, B, C, or D) was applied with a mascara applicator, e.g. the Telescopic® applicator by L'OREAL, then a powder was applied, the powder being constituted by 100% Colorona Glitter Bronze nacres by Merck. The powder was applied using a foam endpiece, e.g. the applicator of the Palette Yeux by Lancôme.

The results were as follows:

| | Immediate | | Staying | |
|---|---|---|---|---|
| | adhesion | Ease of | power | Ease of |
| | of the | applying | of the | removing |
| | powder | makeup | powder | makeup |
| Composition A | Poor | Easy | Poor | Easy |
| Composition B | Average | Easy | Average | Easy |
| Composition C | Very good | Difficult | Very good | Very |
| | | (sticks | | difficult |
| | | eyelashes | | |
| | | together) | | |
| Composition D | Very good | Easy | Good | Easy |

The shine was measured on 18 hair specimens to which makeup had been applied individually (integral of the reflectance as a function of angle after being illuminated by a light source oriented at 45°).

| | Shine of | Shine after | Shine after |
|---|---|---|---|
| | the hair | applying the | applying the |
| | specimen | base | base + nacres |
| Composition A | 1 | 1.2 | 1.9 |
| Composition B | 1 | 2.6 | 2.5 |
| Composition D | 1 | 1.1 | 4.1 |

### Example 2

The following adhesive composition was applied to the eyelashes:

| | |
|---|---|
| Gel-Tac 600D** | 10 |
| Hydroxyethylcellulose | 3 |
| (HEC) | 87 |
| Water | |

**Adhesive particles in aqueous solution sold by Advanced Polymer International (44% dry extract).

The adhesive composition was applied with a Telescopic® mascara brush by L′OREAL.

This composition formed a discontinuous film on the eyelashes.

A powder composition was applied having the following formulation:

| | |
|---|---|
| Colorona bronze glitter nacres (Merck) | 100 |

The powder composition was applied using a foam endpiece for example, similar to the foam endpiece found in Palette Yeux by Lancôme.

A particularly attractive deposit of particles was obtained, as shown in FIG. 2.

### Example 3

The adhesive composition in Example 2 was applied to the eyelashes, followed by a fiber-based composition, Morphotone by TEIJIN, having the following formulation:

| | |
|---|---|
| Morphotone GM100C600 (TEIJIN FIBERS) | 100 |

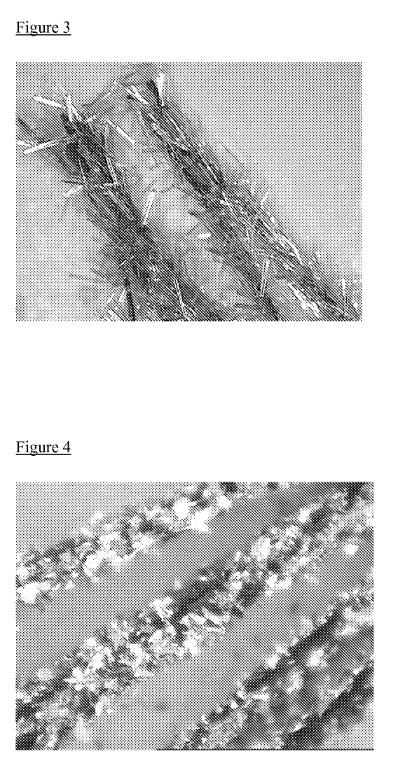

The result shown in FIG. 3 was obtained.

### Example 4

The adhesive composition in Example 2 was applied to the eyelashes, followed by a reflective-particle based composition (Metashine by NSG), having the following formulation:

| | |
|---|---|
| METASHINE ME 2040 PS by Nippon Sheet Glass | 100 |

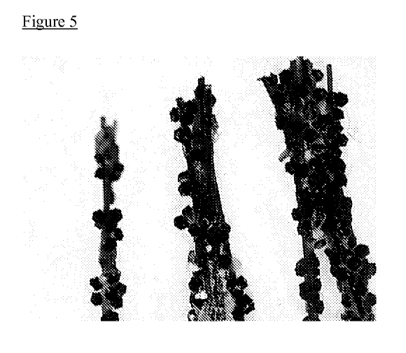

The result shown in FIG. 4 was obtained.

### Example 5

The adhesive composition in Example 2 was applied to the eyelashes, followed by a black-particle based composition by Glitterex, having the following formulation:

| | |
|---|---|
| 150 μm black flakes (BLACK 0.006 HEX COSMETA | 100 |
| GEM by Glitterex) | |

The result shown in FIG. 5 was obtained.

The invention is not limited to the embodiments described. The invention also makes it possible to treat other keratinous fibers, such as bristles, the hair, the eyebrows . . . . Exceptional shine effects may be obtained by treating the hair or portions of hair with a pigment having color and/or shine that contrast with the original color. In particular, it is possible to use pigments having color that is lighter than the color of the original hair. By means of the invention, the effect is so visible that it is not necessary to treat a large amount of hair in order to obtain a satisfactory effect.

It is thus possible to treat the eyelashes, bristles, the hair, or other fibers so as to make them less visible, e.g. so as to make hairs and down on the face, arms, legs . . . disappear. It is also possible to treat epilated zones so as to make hairs that grow back disappear.

### Example 6

Composition D in Example 1 was applied to the hair. About 1 hair in 50 was treated in this way. A very attractive visual effect was obtained.

### Example 7

The pigments used in the second composition were flesh color.

The test was performed on the top of a forearm that presented hair that was quite visible (brown hairs of about 7 millimeters (mm) to 1 centimeter (cm)).

The hair disappeared.

### Example 8

The same test as above was performed, with the difference that the method was applied to armpits that had been epilated 10 days beforehand.

The hairs were no longer visible.

The expression “comprising a” should be understood as being synonymous with “comprising at least one”.

1. A method of applying makeup to human keratinous fibers, said method comprising:
applying on the fibers an adhesive composition that forms a discontinuous deposit on the fibers; and
after applying the adhesive composition, applying on the fibers, a loose powder composition.

2. A method according to claim 1, wherein the adhesive composition includes a pressure-sensitive adhesive.

3. A method according to claim 1, wherein the adhesive composition comprises adhesive particles that are not fluid.

4. A method according to claim 1, wherein the adhesive composition comprises an adhesive selected from:
acrylic polymers, rubber-based or styrene copolymer-based pressure-sensitive adhesives, polymers of urethane, polyurethanes, cross-linked silicones, polymers of ethylene-vinyl acetate, styrene-based block copolymers or natural gums, and polymers of chloroprene, butadiene, isoprene, and/or neoprene.

5. A method according to claim 4, wherein the adhesive is selected from:
natural rubber (poly(cis-1,4-isoprene)), methyl-methacrylate-isoprene graft copolymers, styrene butadiene copolymers, butyl rubber, acrylonitrile-butadiene rubber, styrene-isoprene block copolymers, polybutadiene, ethylene-butylene block copolymers, and polychloroprene;
acrylic acid-based block or statistical copolymers, alkyl acrylates and alkyl methacrylates, and copolymers of such acrylics with ethylene and vinyl acetate;
butyl acrylate, butyl methacrylate, and acrylic acid copolymers; and
poly(2-ethylhexylacrylate).

6. A method according to claim 1, wherein the loose powder composition includes particles in the form of reflective flakes.

7. A method according to claim 6, wherein the reflective-flake particles are:
made out of a single material that is optically uniform, having at least two parallel faces.

8. A method according to claim 1, wherein a particle content in the loose powder composition is greater than or equal to 30% by weight.

9. A method according to claim 1, wherein a size of the particles of the loose powder composition is greater than or equal to 20 μm.

10. A method according to claim 1, wherein a greatest dimension of the particles of the loose powder composition ranges from 10 μm to 200 μm.

11. A method according to claim 1, wherein the loose powder composition comprises particles having at least one plane face or having a radius of curvature that is greater than or equal to 60 μm.

12. A method according to claim 1, wherein the keratinous fibers comprise eyelashes.

13. A method according to claim 1, wherein the adhesive composition comprises adhesive particles having an average size ranging from 1 μm to 250 μm.

14. A cosmetic kit comprising:
an adhesive composition configured to form a discontinuous film of adhesive particles on the keratinous fibers;
a powder composition;
a first applicator member configured to apply the adhesive composition to the keratinous fibers; and
a second applicator member configured to apply the powder composition to the keratinous fibers.

15. A cosmetic kit according to claim 14, wherein an average size of the adhesive particles ranges from 1 μm to 250 μm.

16. A method according to claim 1, wherein the adhesive composition comprises an adhesive selected from acrylate and methacrylate copolymers.

17. A method according to claim 1, wherein the adhesive composition comprises an adhesive selected from styrene-isoprene-styrene (SIS) and styrene-butadiene-styrene (SBS) copolymers.

18. A method according to claim 6, wherein the reflective-flake particles have a layered structure and include at least two layers of material having different optical properties.

19. A method according to claim 18, wherein the flakes comprise pigments having a core/shell structure or pigments having a multilayer structure without a substrate.

20. A method according to claim 18, wherein the flakes comprise aluminum, bronze, silica, synthetic mica, glass, or transparent-effect pigments.

21. A method according to claim 18, wherein the flakes are formed of natural or synthetic mica coated with at least one layer of metal oxide, of iron, of tin, of chromium, or from barium sulfate, MgF₂, CrF₃, ZnS, ZnSe, SiO₂, Al₂O₃, MgO, Y₂O₃, SeO₃, SiO, HfO₂, ZrO₂, CeO₂, Nb₂O₅, Ta₂O₅, MoS₂, or titanium mica coated with iron oxide, mica coated with bismuth oxychloride, titanium mica coated with chromium oxide, or titanium mica coated with an organic colorant.

22. A method according to claim 18, wherein the flakes comprise nacre pigments based on bismuth oxychloride, particles of mica on the surface of which at least two successive layers of metal oxides and/or organic coloring materials have been superposed, reflective particles having a multilayer structure with at least one layer of uniform thickness or reflective particles having a multilayer structure and having a natural or synthetic substrate, or particles comprising a substrate of borosilicate coated with silver.

23. A method according claim 1, wherein a particle content in the loose powder composition is greater than or equal to 80% by weight.

24. A method according to claim 1, wherein a size of the particles of the loose powder composition is greater than or equal to 40 μm.

25. A method according to claim 1, wherein the adhesive composition comprises adhesive particles having an average size ranging from 10 μm to 250 μm.

26. A cosmetic kit according to claim 14, wherein an average size of the adhesive particles ranges from 10 μm to 250 μm.
